(19) United States
(12) Patent Application Publication
(10) Pub. No.: US 2024/0148963 A1
(43) Pub. Date: May 9, 2024

(54) SYSTEMS AND METHODS UTILIZING DATA FOR ENTERAL NUTRITION

(71) Applicant: Luminoah, Inc., Charlottesville, VA (US)

(72) Inventors: Joseph Neal Piper, Charlottesville, VA (US); Brian Bergeron, Charlottesville, VA (US); Martin Eric Weiner, Charlottesville, VA (US); Andrew DeHennis, Charlottesville, VA (US); Hill Johnson, Charlottesville, VA (US); James Landon Gilkey, Charlottesville, VA (US)

(21) Appl. No.: 18/387,614

(22) Filed: Nov. 7, 2023

Related U.S. Application Data
(60) Provisional application No. 63/382,802, filed on Nov. 8, 2022.

(51) Int. Cl. A61M 5/142; A61B 5/00; G16H 10/60; G16H 15/00; G16H 20/60

(52) U.S. Cl. CPC A61M 5/142; A61B 5/0002; A61B 5/6801; A61B 5/7435; G16H 10/60; G16H 15/00; G16H 20/60; A61B 2562/0219; A61B 2562/0233; A61M 2005/14208

(57) ABSTRACT

A wearable fluid delivery system is part of a networked system to collects, transmits, stores, and analyzes data related to enteral nutrition. A method includes inputting patient data; generating a nutrition plan; implementing the nutrition plan with a wearable nutrition device; detecting a parameter with the wearable nutrition device; and generating an updated nutrition plan based on the parameter.

### RELATED APPLICATIONS

This application claims priority to and the benefit of U.S. Provisional Patent Application No. 63/382,802, filed on Nov. 8, 2022, which is incorporated herein by reference in its entirety for all purposes.

### BACKGROUND

Enteral nutrition, or tube feeding, is a process that delivers nutrition directly to the stomach or small intestine in place of traditional oral feeding. If a patient is receiving treatment outside of a hospital setting, the process is referred to as Home Enteral Nutrition (HEN). A 2013 study indicates that as many as 250,000 adults and 190,000 children currently require HEN as a part of their medical treatment in the United States. Currently, the leading conditions that indicate a need for HEN include cancer, nonmalignant respiratory disease, and neurological disorders. Enteral nutrition currently requires an array of medical resources and technologies including doctor assessment, a nutrition plan prescribed by a nutrition support team, a surgically implanted gastrostomy tube, a delivery system, tubing sets, and a nutritional formula. Medical patients for whom oral feeding is not allowable or sufficient commonly benefit from prescribed enteral nutrition. This form of therapy delivers nutrition directly to a patient's gastrointestinal tract (GI) through man-made tubes that are placed into the GI tract. In order to access any portion of the patient's GI tract, the placed tubes must enter the patient's body through incisions created in the patient's abdominal wall or through existing body cavities such as the nasal cavity.

The distal end of any such tube is placed in the GI tract, while the proximal end of any such tube remains outside of the patient's body, permitting the proximal end to interface with enteral nutrition delivery technology. Surgically implanted tubes are generally indicated for long-term enteral nutrition needs while nasally placed tubes are indicated for short-term (less than two months) needs or when a patient is not healthy enough to tolerate surgery. Commonly, gastrostomy tubes are placed one of three ways: (1) surgically, through an open procedure or laparoscopically, (2) endoscopically, or (3) radiologically with a percutaneous insertion procedure.

Malnutrition and dysphagia are increasing, especially in chronic disease patients and elderly people. The occurrence of malnutrition is high in patients with chronic illnesses like cancer, neurological disorders, heart failure, and COPD, and increases with age as well. The prevalence of various cancers, especially gastric, head and neck/throat, and esophageal cancers, is growing globally, correlating to a rise in the need for enteral feeding in some oncology patients. Also, there is an increase in new markets where enteral feeding is playing a role for the first time. These include areas such as sports medicine and athletic training, pregnant women who suffer from hyperemesis gravidarum, and treatment for bulimia/anorexia conditions.

### SUMMARY

The disclosure provides, in one aspect, a method comprising inputting patient data; generating a nutrition plan; implementing the nutrition plan with a wearable nutrition device; detecting a parameter with the wearable nutrition device; and generating an updated nutrition plan based on the parameter.

In some embodiments, the method further includes implementing the updated nutrition plan with the wearable nutrition device.

In some embodiments, patient data includes gender, date of birth, height, weight, activity level, goal weight, organic preference, time preference, or any combination thereof.

In some embodiments, the parameter includes nutritional intake.

In some embodiments, the method further includes generating a nutrition report, wherein the nutrition report includes a historical view of patient nutrition.

In some embodiments, the nutrition report includes a score, and wherein the score is determined based on calories consumed, activity level, water consumed, micronutrition, patient demographic information, or any combination thereof.

In some embodiments, the nutrition report includes a score, and wherein the score is determined based on calories consumed, activity level, water consumed, micronutrition, and patient demographic information.

In some embodiments, the nutrition report is accessible via a network.

In some embodiments, generating the nutrition plan is based on a database of parameters from a plurality of users.

In some embodiments, the nutrition plan includes a custom formulation.

In some embodiments, the nutrition plan includes a caloric goal, a plurality of feed times, a flow rate, a formulation, or any combination thereof.

In some embodiments, the nutrition plan includes a caloric goal, a plurality of feed times, a flow rate, and a formulation.

In some embodiments, the patient data is collected by a wearable device.

In some embodiments, the method further includes detecting a second parameter with a wearable device; and generating the updated nutrition plan based on the second parameter.

In some embodiments, implementing the nutrition plan includes operating a motor to drive a pump of the wearable nutrition device, whereby a fluid is pumped from a container to a patient.

In some embodiments, detecting the parameter includes detecting an acceleration with an accelerometer positioned within the wearable nutrition device.

In some embodiments, detecting the parameter includes detecting an orientation with an accelerometer positioned within the wearable nutrition device.

In some embodiments, detecting the parameter includes receiving a user input from a user interface on the wearable nutrition device, and wherein the user input is in response to nausea, bloating, discomfort, regurgitation, dislodgment, an error message, or any combination thereof.

In some embodiments, detecting the parameter includes detecting with a gastric access device a pH, a microbiome, a gas, or any combination thereof.

In some embodiments, the method further includes displaying the parameter on a display of the wearable nutrition device.

The disclosure provides, in one aspect, a method comprising detecting a parameter with a wearable nutrition device; wirelessly transmitting the parameter from the wearable nutrition device to a user device; uploading the parameter from the user device to a network; storing the parameter in a database on the network; and retrieving the parameter from the database at a terminal connected to the network.

In some embodiments, detecting the parameter includes detecting orientation with an accelerometer.

In some embodiments, detecting the parameter includes detecting an optical property with an optical sensor.

In some embodiments, detecting the parameter includes receiving a user input from a user interface.

In some embodiments, the user input is in response to nausea, bloating, discomfort, regurgitation, dislodgment, or an error message.

In some embodiments, detecting the parameter includes wirelessly detecting an identifying parameter.

In some embodiments, a wireless tag is coupled to a disposable portion of the wearable nutrition device and a wireless receiver is coupled to a reusable portion of the wearable nutrition device, and wherein the wireless receiver wirelessly detects the identifying parameter from the wireless tag.

In some embodiments, the identifying parameter includes nutritional information, run time information, or recall information.

In some embodiments, detecting the parameter includes detecting steps with an accelerometer or a pedometer.

In some embodiments, the method further includes determining an updated control variable based on the detected parameter.

In some embodiments, the updated control variable is an updated flow rate.

In some embodiments, the method further includes wirelessly transmitting the updated control variable to the wearable nutrition device from the network.

In some embodiments, the method further includes generating a nutrition report based on retrieving the parameter from the database.

In some embodiments, the wearable nutrition device is worn by a patient, the user device is utilized by the patient, and the terminal is utilized by a physician.

In some embodiments, the method further includes detecting a second parameter with a wearable device; and wirelessly transmitting the second parameter from the wearable device to the user device.

In some embodiments, the method further includes uploading the second parameter from the user device to the network; and storing the second parameter in a second database on the network.

In some embodiments, the method further includes displaying the parameter on a display of the wearable nutrition device.

In some embodiments, the method further includes displaying the parameter on the user device and on the terminal.

In some embodiments, detecting the parameter includes detecting pH, microbiome, and/or gas with a gastric access device.

Other aspects of the disclosure will become apparent by consideration of the detailed description and accompanying drawings.

### BRIEF DESCRIPTION OF THE DRAWINGS

FIG. 1 is schematic of a networked system including a wearable nutrition device, a user device, and a terminal.

FIG. 2 is flowchart for a method for initializing a provider account.

FIG. 3 is a flowchart for a method for generating a nutrition plan.

FIG. 4 is a flowchart for a method for implementing and modifying a nutrition plan.

FIG. 5 represents a nutrition report.

FIG. 6 is a graph of BMI tracking and maintenance with BMI shown as a function of age.

FIG. 7 is a graph of daily feeding history breakdowns.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not. Further, unless otherwise required by context, singular terms shall include pluralities and plural terms shall include the singular.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the terms “processor” and “central processing unit” or “CPU” are used interchangeably and refer to a device that is able to read a program from a computer memory (e.g., ROM or other computer memory) and perform a set of steps according to the program. As used herein, the term “processor” (e.g., a microprocessor, a microcontroller, a processing unit, or other suitable programmable device) can include, among other things, a control unit, an arithmetic logic unit (“ALC”), and a plurality of registers, and can be implemented using a known computer architecture (e.g., a modified Harvard architecture, a von Neumann architecture, etc.). In some embodiments the processor is a microprocessor that can be configured to communicate in a stand-alone and/or a distributed environment, and can be configured to communicate via wired or wireless communications with other processors, where such one or more processor can be configured to operate on one or more processor-controlled devices that can be similar or different devices.

As used herein, the term “memory” is any memory storage and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processor can be connected to the memory and execute software instructions that are capable of being stored in a RAM of the memory (e.g., during execution), a ROM of the memory (e.g., on a generally permanent bases), or another non-transitory computer readable medium such as another memory or a disc. In some embodiments, the memory includes one or more processor-readable and accessible memory elements and/or components that can be internal to the processor-controlled device, external to the processor-controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the processor can be configured to retrieve from the memory and execute, among other things, instructions related to the processes and methods described herein.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks, whether local or distant (e.g., cloud-based).

“About” and “approximately” are used to provide flexibility to a numerical range endpoint by providing that a given value may be “slightly above” or “slightly below” the endpoint without affecting the desired result.

The term “coupled,” as used herein, is defined as “connected,” although not necessarily directly, and not necessarily mechanically. The term coupled is to be understood to mean physically, magnetically, chemically, fluidly, electrically, or otherwise coupled, connected or linked and does not exclude the presence of intermediate elements between the coupled elements absent specific contrary language.

As used herein, the term “in electronic communication” refers to electrical devices (e.g., computers, processors, etc.) that are configured to communicate with one another through direct or indirect signaling. Likewise, a computer configured to transmit (e.g., through cables, wires, infrared signals, telephone lines, airwaves, etc.) information to another computer or device, is in electronic communication with the other computer or device. As used herein, the term “transmitting” refers to the movement of information (e.g., data) from one location to another (e.g., from one device to another) using any suitable means.

As used herein, the term “network” generally refers to any suitable electronic network including, but not limited to, a wide area network (“WAN”) (e.g., a TCP/IP based network), a local area network (“LAN”), a neighborhood area network (“NAN”), a home area network (“HAN”), or personal area network (“PAN”) employing any of a variety of communications protocols, such as Wi-Fi, Bluetooth, ZigBee, etc. In some embodiments, the network is a cellular network, such as, for example, a Global System for Mobile Communications (“GSM”) network, a General Packet Radio Service (“GPRS”) network, an Evolution-Data Optimized (“EV-DO”) network, an Enhanced Data Rates for GSM Evolution (“EDGE”) network, a 3GSM network, a 4GSM network, a 5G New Radio, a Digital Enhanced Cordless Telecommunications (“DECT”) network, a digital AMPS (“IS-136/TDMA”) network, or an Integrated Digital Enhanced Network (“iDEN”) network, etc.

As used herein, the term “subject” or “patient” refers to any animal (e.g., a mammal), including, but not limited to, humans, non-human primates, companion animals, livestock, equines, rodents, and the like, which is to be the recipient of a particular treatment. Typically, the terms “subject” and “patient” are used interchangeably herein in reference to a human subject.

As used herein, the term “accelerometer” is a sensor (e.g., a hardware component) that can determine orientation (e.g., orientation of a user and/or the device at any time). As used herein, the term “pedometer” is a sensor (e.g., a hardware component) that can determine the number of steps and/or the amount of activity of a user. An accelerometer can be used as a pedometer.

As used herein, the term “optical sensor” is a sensor that can determine and monitor the flow of fluid through a system and recognize any occlusions, air bubbles, etc. The optical sensor can verify the system is moving fluid through as expected.

As used herein, the term “radio frequency identification” (RFID) is a contactless one-way communication method at varying distance. “Near field communication” (NFC) allows for two-way communication and can require an action by the user.

As used herein, the term “nutrition report” is a collection of data displayed for a user. The nutrition report is a resource for healthcare providers and patients that displays a variety of important information tailored towards, for example, treatment goals, treatment progress, and other physiological metric summaries. The nutrition report uses data collected by the systems.

As used herein, the term “event” is an occurrence that a patient inputs into the system to characterize or add context to their feeding experience. An event may include a feed related symptoms including, but not limited to, nausea, bloating, or discomfort. An event may include an adverse event including, but not limited to, regurgitation, dislodgement, or receiving an error message.

As used herein, the term “body mass index” (BMI) is a value derived from the mass and height of a person. Often used as metric for defining the success of an enteral nutrition program or plan.

As used herein, the term “automated nutrition plan” is an optimized feeding regimen for a patient that is updated based on monitoring and data collection of system parameters, physiological inputs, and/or real-time monitoring of users.

As used herein, the term “score” or “Lumiscore” or “NutriNoah Score” is a scoring system (e.g., 1-10, 1-100, or A-F) that assesses the nutritional status, progress, and outlook for an individual patient.

### 2. WEARABLE NUTRITION DEVICE

A wearable fluid delivery system is described in U.S. patent application Ser. No. 17/478,905, filed Sep. 18, 2021, the entire contents of which are incorporated herein by reference. Also, a wearable fluid delivery system providing regimen-predictive analytics is described in U.S. patent application Ser. No. 17/645,181, filed on Dec. 20, 2021, the entire contents of which are incorporated herein by reference.

In some embodiments, the wearable nutrition device 10 is a wearable fluid delivery system with a single-use portion 14 including a tube 18, a pump 22, and a nutrition container 26 (e.g., a pouch) with a fluid. The wearable fluid delivery system further includes a module 30 (e.g., a main module) including a processor, a battery, and an electric motor with an output member. The processor is configured to operate the electric motor. The single-use device 14 is releasably coupled to the module 30 such that the electric motor is coupled to the pump when the single-use portion 14 is coupled to the module 30. The output member of the electric motor drives the pump 22 to cause the fluid to travel through the tube 18. The wearable fluid delivery system or any component thereof may be interchangeably referred to herein as a “wearable nutrition device.”

The wearable nutrition device 10 provides an opportunity for detailed and individualized data collection around enteral nutrition patients that conventional systems cannot provide. Some ways the system collects, stores, transmits, and analyze data are provided below, with additional examples available in Example 1.

The wearable nutrition device 10 includes an accelerometer. The accelerometer detects the orientation and activity level of a patient. In some embodiments, activity tracking includes general motion, active motion (e.g., burning calories, walking, jogging), number of steps taken, and user position (e.g. laying down, sitting, standing). In some embodiments, an orientation of a display 34 on the module 30 is adjusted automatically based on the detected user position so the user can read the display at the correct angle.

In some embodiments, the fluid flow rate is lowered in response to detecting the patient is in a lying down position (e.g., with head propped at least 30 degrees). In some embodiment the fluid flow rate is increased in response to detecting the patient is in a sitting up or standing up position. A sustained supine position (with the head-of-bed flat) increases gastro-esophageal reflux and the probability of aspiration. In other words, the wearable nutrition device 10 automatically adjusts the flow rate of fluid through the system and to the patient based on the detected orientation of the patient.

In some embodiments, the fluid flow rate is lowered in response to detecting the patient activity level is high (e.g., fast walking, jogging, running) In some embodiments, the fluid flow rate is increased in response to detecting the patient activity level is low (e.g., not active, sedentary). In other words, the wearable nutrition device 10 automatically adjusts the flow rate of fluid through the system and to the patient based on the detected activity level of the patient. In some embodiments, users input the current activity level on user device upon first use for baseline data. As the patient wears the fluid delivery system, activity (e.g., total steps and calories burned) can be tracked and trends will be formulated based on day/week/month/quarter/year, for example.

The wearable nutrition device 10 includes an optical sensor. The optical sensor can detect the type of fluid being pumped through the system. In some embodiments, the optical sensor detects the light refraction based on the material or fluid properties present within the system. In some embodiments, the optical sensor is able to differentiate and detect nutrition (e.g., a dark color), hydration (e.g., water), and air. This enables the system to determine whether and when it is delivering nutrition or hydration and to automate the process of tallying the associated totals. This can be used in real-time (e.g., active patient monitoring) in addition to being used longitudinally for the creation of a nutrition algorithm based on user data (e.g., when, how much, and how often users are switching formula types, formula to water, etc.).

The wearable nutrition device 10 includes a user interface configured to receive a user input. Receiving user input allows a user to log or track events (e.g., event reporting). In other words, users can input information into the system related to an event or occurrence (e.g., feed related symptoms including nausea, bloating, or discomfort and/or an adverse event including regurgitation, dislodgement, or error message). In some embodiments, after each feed, the patient gets a notification such as: “How are you feeling?” and selects one of a plurality of pre-selected prompts (e.g., nauseous, vomiting, regurgitation, tired, dizzy, hungry, full, satisfied, happy). These are called events and can be recorded at any time during a feed or after. For example: if patient is reporting negative events related to current nutrition, a provider may choose to change the formulation. If patient is constipated, the provider may recommend additional hydration or recommend a laxative.

The wearable nutrition device 10 includes a RFID and/or a NFC communication system. In some embodiments, the wearable nutrition device 10 includes data exchange between the disposable pump head 22 or the container 26 and the module 30. In some embodiments, the disposable pump head 22 or the container 26 includes a RFID tag and the module 30 includes RFID transmitter and receiver configured to detect the RFID tag. This enables analysis regarding when and how often a new pump head or daily administration sets are used. This also enables making the pump head obsolete after a pre-defined period of time to, for example, reduce bacteria build up in the system. The data exchange between the disposable nutrition pouch and the module also enables collecting the type of nutrition that is about to be infused to the patient and what the nutritional breakdown of that formula is. In some embodiments, system components affected by a recall (e.g., a faulty pump) are identified using the RFID/NFC system.

The wearable nutrition device 10 includes a gastric access device 38 (e.g., a “G-Button”) with sensing capabilities. The gastric access device with sensing provides additional insight into the relationship between the body and feeding. For example, the gastric access device can detect information related to: the pH of the gut, the microbiome of the intestines, or gas or other physiological responses in the stomach to feeding.

As detailed further herein, data detected by the wearable nutrition device 10 is transmitted and stored such that a provider can make adjustments to a patient nutrition plan (e.g., nutrition and hydration) based on various relevant variables, including, but not limited to: age, BMI, activity level, calories burned, tolerance to formula or formula types (e.g., standard, protein-rich, calorie-rich, low electrolytes, fiber-rich, reduced-calorie, peptide based, substrate-enriched, organic, etc.)

### 3. NETWORKED SYSTEM

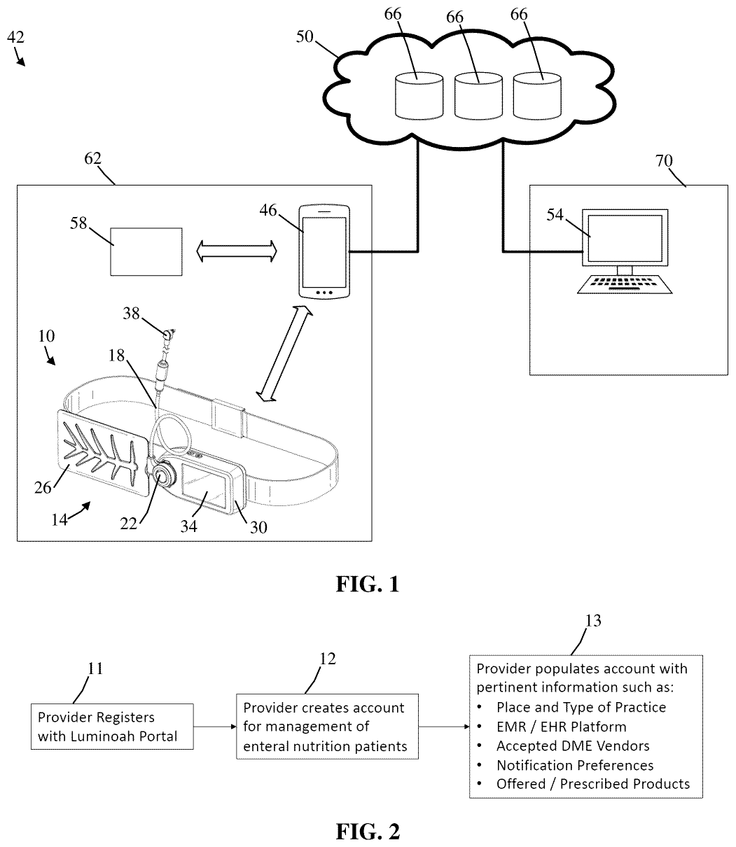

With reference to FIG. 1, a networked system 42 includes the wearable nutrition device 10, a user device 46, a network 50, and a terminal 54.

In some embodiments, the user device 46 is a cellular phone, a tablet, a personal computer, or any other suitable user device. In the illustrated embodiment, the user device 46 is a cellular phone with a display.

In some embodiment, the system 42 further includes a wearable device 58. In some embodiments, the wearable device 58 is a fitness tracker such as a Fitbit or Apple watch.

In the illustrated embodiment, the wearable nutrition device 10, the user device 46, and the wearable device 58 are located on or near a user in a first location 62. In some embodiments, the first location 62 is a wherever the user is located and moves with the user.

In some embodiments, the network 50 is a cloud-based network connected to the internet. In some embodiments, the network 50 includes a plurality of databases 66 for the storage of various data. In some embodiments, data collected from the wearable nutrition device 10 is stored in the database 66. In some embodiments, data collected from the wearable device 58 and/or the user device 46 is stored in the database 66. The databases 66 may include information collected from a plurality of patients and providers.

In some embodiments, the networked system is integrated with a health informatics system (e.g., Apple Health Kit). As such, when a user is wearing the wearable nutrition device 10 the accelerometer and/or pedometer can track activity levels, but when the user is not wearing the wearable nutrition device 10, this data can instead be captured through the accelerometer and/or pedometer of the wearable device 58 (e.g., a smart watch or smart phone). In some embodiments, when the wearable nutrition device 10 is not worn, the patient activity is tracked through the patient's phone. This also allows for distinguishing between activity levels when wearing the system 10 versus when not wearing the system 10, which may be utilized in, for example, a quality of life study. When conducting clinical research, this can be used to determine how active the patient is when wearing and not wearing the system. This information will improve comparisons with conventional systems.

In some embodiments, the terminal 54 is a computer, a mobile phone, a tablet, etc. connected to the network 50. The terminal 54 can be any suitable network connected device that enables access and manipulation of the databases. In the illustrated embodiment, the terminal 54 is located at a second location 70. In some embodiments, the second location 70 is spaced from or remote from the first location 62. For example, the first location 62 may include a user's home and the second location 70 may include a physician's office. Both the first location 62 and the second location 70 are connected to the network 50.

In the illustrated embodiment, data is transmitted from the module 30 of the wearable nutrition device 10 to the user device 46 (e.g., a mobile application on a cell phone). The data on the user device 46 is then transmitted (e.g., via WiFi) to the network 50 and stored in databases 66. In some embodiments, the raw data is coded to each system serial number to protect patient data. In some embodiments, data and control instructions flow bidirectionally between the wearable nutrition device 10 and the network 50. In other words, the wearable nutrition device 10 can send data to the network 50, and the network 50 can send data or updated control instructions to the wearable nutrition device 10.

### 4. NUTRITION REPORT

The nutrition report (“a report”) is a resource for providers to provide routine monitoring following the standard of care to include progress toward nutritional and medical treatment goals, identify potential problems, and take action to improve outcomes. The report follows the standard of care for enteral nutrition management and is developed using data generated and collected from the networked system 42 including provider and user inputs.

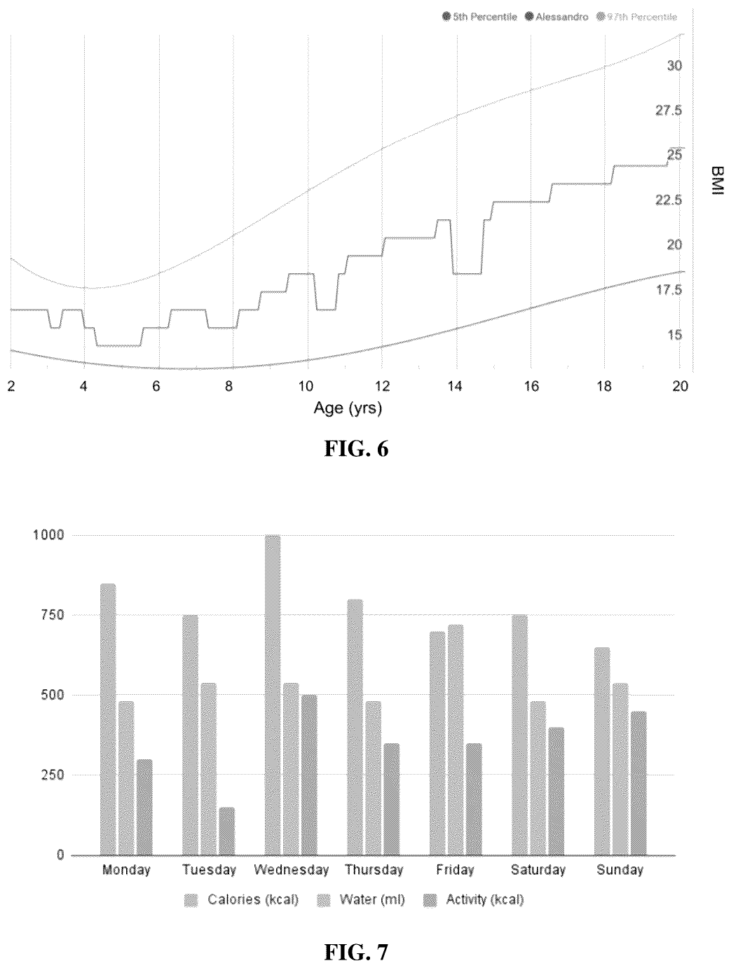

A nutrition report is generated for each patient. If a patient shares data with a provider or caregiver, the provider or caregiver will also receive the report. With reference to FIG. 5, an example nutrition report is illustrated. The nutrition report is designed to encapsulate in a single document the important information for each of the primary stakeholders in the continuum of care E.g., physician, DME vendor, and user). Some physician-centric information includes patient identifying information 74. Some DME vendor-centric information 78 includes a DME provider, shipping address, phone number, and primary contact. Some user-centric information includes graphical representations of feeding history including goal tracking 82, dietary breakdown 86, BMI tracking (FIG. 6), and daily totals (FIG. 7).

In some embodiments, the nutrition report includes a score 90 (e.g., a Lumiscore or NutriNoah score) that provides a quantitative rating of the user's nutritional status based on the collected data. In some embodiments, the score is based on calories consumed, activity level, water consumed, macronutrient breakdown, and demographic information. The score is specifically tailored to the patient, meaning that the inputs into the score can be weighted differently. For example, a child who is healthy outside of the need for enteral nutrition will have their activity level impact their score more than a user who may be wheelchair bound.

In some embodiments, a cloud-based portal enables providers to build out a customized nutrition plan and track progress based on actual data generated by the system with user inputs related to potential problems through events. The report will ensure providers are following the standard of care and ensure intended health benefits of enteral nutrition are meeting met. Intended health benefits include but are not limited to: provide adequate nourishment when there is unintended, reversible weight loss; improve clinical status; enhance patient comfort and quality of life; and to prolong life. The online portal for physicians will provide an expanded view of patient data that allows them to curate the information to be the most useful to their patient. For example, a chemotherapy patient will be focused on maintaining weight and BMI, and the physician can create a report that is focused on the factors contributing to BMI/weight gain. As another example, a patient with a metabolic disease will want granular insight into the macronutrient breakdown of their diet, and the physician can create a report that is focused on the factors contributing to their nutritional breakdown

At any time, the nutrition report and its accompanying information may be available to users or providers through a cloud-based system. A cloud-based system will be used to make a clinician/user portal that will allow real-time access to information stored in the cloud for the nutrition report. Data from the user's wearable nutrition device 10 will transmit (e.g., via Bluetooth) to the user's smart phone 46, and the data will then transfer to the cloud 50 which can be accessed by providers, users, and caregivers that are granted access at a remote terminal 54.

### 5. CUSTOM FORMULAS

In some embodiments, a custom nutrition formula is developed based on user reports, activity level, and self-reported events. The custom nutrition formula is formulated specifically for patients needs and can be prefilled in nutrition pouches. A specific enteral nutrition formulary can be established based on patient population and estimated nutrient needs rather than specific diagnosis. For example, historical data collected from users (i.e. feeding regimens, treatment settings, dietary breakdown) can be used to create personalized nutritional formulations that best serve a patient based on their need. As another example, historical data from users (i.e. feeding regimens, treatment settings, dietary breakdown) can be used to prospectively recommend certain formulations for users that have worked for similar types of patients previously.

In some embodiments, the custom nutrition formula is based on information collected from the gastric access device 38 (e.g., “G-Button”). Information collected from the stomach or gut can be applied to the creation of a formula to supplement users with: good probiotics; macronutrient needs; medications (for ulcers, or other stomach issues); new route of administration; and possible allergens that were previously undetected.

### 6. AUTOMATION ALGORITHM

Data collected from an individual patient and or data collected from a plurality of patients can be applied to create an automated feeding algorithm. Collected data is used in artificial intelligence and machine learning to make automated adjustments to a nutrition plan (e.g., automated adjustments to flow rate) in real-time. In some embodiments, the machine learning model is an artificial neural network. In addition, the wearable nutrition device 10 can respond in real-time to changes in the environment, activity, or other factors that may affect the needs of an enteral nutrition patient.

In some embodiments, the automated feeding algorithm utilize a variety of input information such as, but not limited to: age, BMI, activity levels, calories burned, tolerance to formula or formula types (Standard, protein-rich, calorie-rich, low electrolytes, fiber-rich, reduced-calorie, peptide-based, substrate-enriched, organic, and other). The user's activity level will help providers determine which nutrition brand to use and how many calories to deliver to maintain, lower, or increase BMI to provide better health outcomes.

The automated feeding algorithm can also be utilized when onboarding a new patient. For example, a provider or patient can input the name of the nutrition used and the automated feeding algorithm calculates calories and contents (carbohydrates, protein, fats) automatically. In some embodiments, the automated feeding algorithm is executed on the network. In other embodiments, the automated feeding algorithm is executed locally on the wearable nutrition device 10 (e.g., a processor in the module 30).

In one embodiment, a method comprises: inputting patient data; generating a nutrition plan; implementing the nutrition plan with a wearable nutrition device; detecting a parameter with the wearable nutrition device; and generating an updated nutrition plan based on the parameter.

In some embodiments, the method further includes implementing the updated nutrition plan with the wearable nutrition device.

In some embodiments, the patient data includes gender, date of birth, height, weight, activity level, goal weight, organic preference, time preference, or any combination thereof.

In some embodiments, the parameter includes nutritional intake.

In some embodiments, the method further includes generating a nutrition report, wherein the nutrition report includes a historical view of patient nutrition.

In some embodiments, the nutrition report includes a score, and wherein the score is determined based on calories consumed, activity level, water consumed, micronutrition, patient demographic information, or any combination thereof.

In some embodiments, the nutrition report includes a score, and wherein the score is determined based on calories consumed, activity level, water consumed, micronutrition, and patient demographic information.

In some embodiments, the nutrition report is accessible via a network.

In some embodiments, generating the nutrition plan is based on a database of parameters from a plurality of users.

In some embodiments, the nutrition plan includes a custom formulation.

In some embodiments, the nutrition plan includes a caloric goal, a plurality of feed times, a flow rate, a formulation, or any combination thereof.

In some embodiments, the nutrition plan includes a caloric goal, a plurality of feed times, a flow rate, and a formulation.

In some embodiments, the patient data is collected by a wearable device (e.g., a Fitbit or Apple watch).

In some embodiments, the method further includes detecting a second parameter with a wearable device; and generating the updated nutrition plan based on the second parameter.

In some embodiments, implementing the nutrition plan includes operating a motor to drive a pump of the wearable nutrition device, whereby a fluid is pumped from a container to a patient.

In some embodiments, detecting the parameter includes detecting an acceleration with an accelerometer positioned within the wearable nutrition device.

In some embodiments, detecting the parameter includes detecting an orientation with an accelerometer positioned within the wearable nutrition device.

In some embodiments, detecting the parameter includes receiving a user input from a user interface on the wearable nutrition device, and wherein the user input is in response to nausea, bloating, discomfort, regurgitation, dislodgment, an error message, or any combination thereof.

In some embodiments, detecting the parameter includes detecting with a gastric access device a pH, a microbiome, a gas, or any combination thereof.

In some embodiments, the method further includes displaying the parameter on a display of the wearable nutrition device.

### 7. METHODS AND SYSTEMS

As disclosed herein, the wearable nutrition device 10 and the networked system 42 provide for collection, application, visualization, and reporting of user or patient data. The enteral nutrition provided by the wearable nutrition device 10 is consistent with user and provider created goals for therapy.

The disclosure provides a method comprising: detecting a parameter with a wearable nutrition device; wirelessly transmitting the parameter from the wearable nutrition device to a user device; uploading the parameter from the user device to a network; storing the parameter in a database on the network; and retrieving the parameter from the database at a terminal connected to the network.

In some embodiments, detecting the parameter includes detecting orientation with an accelerometer.

In some embodiments, detecting the parameter includes detecting an optical property with an optical sensor.

In some embodiments, detecting the parameter includes receiving a user input from a user interface (e.g., event reporting).

In some embodiments, the user input is in response to nausea, bloating, discomfort, regurgitation, dislodgment, or an error message.

In some embodiments, detecting the parameter includes wirelessly detecting an identifying parameter (e.g., via RFID or NFC communication). In some embodiments, a wireless tag is coupled to a disposable portion of the wearable nutrition device (e.g., a pump head) and a wireless receiver is coupled to a reusable portion of the wearable nutrition device (e.g., the module), and wherein the wireless receiver wirelessly detects the identifying parameter from the wireless tag.

In some embodiments the identifying parameter includes nutritional information, run time information, or recall information.

In some embodiments, detecting the parameter includes detecting steps with an accelerometer or a pedometer.

In some embodiments, the method further includes determining an updated control variable based on the detected parameter.

In some embodiments, the updated control variable is an updated flow rate.

In some embodiment, the method includes wirelessly transmitting the updated control variable to the wearable nutrition device from the network.

In some embodiments, the method further includes generating a nutrition report based on retrieving the parameter from the database.

In some embodiments, the wearable nutrition device is worn by a patient, the user device is utilized by the patient, and the terminal is utilized by a physician.

In some embodiments, the method further includes detecting a second parameter with a wearable device (e.g., a Fitbit or Apple watch); and wirelessly transmitting the second parameter from the wearable device to the user device.

In some embodiments, the method further includes uploading the second parameter from the user device to the network; and storing the second parameter in a second database on the network.

In some embodiments, the method further includes displaying the parameter on a display of the wearable nutrition device.

In some embodiments, the method further includes displaying the parameter on the user device and on the terminal.

In some embodiments, detecting the parameter includes detecting pH, microbiome, and/or gas with a gastric access device.

In some embodiments, detecting the parameter includes capacitive sensing and sensing bulk material properties using detected changes resonant frequency for a radio frequency resonant circuit.

In some embodiments, detecting the parameter includes sensing using sound waves to probe material properties.

The disclosure provides a method for on boarding a new patient or user including inputting patient data and generating a nutrition plan. In some embodiments, when therapy is initiated, the provider logs into a portal from the terminal 54 and walks through a series of questions generated from the portal with the patient to create a person-specific enteral nutrition care plan. In some embodiments, the questions include: name, gender, date of birth, height, weight, location, MRN. Other factors considered when inputting patient data may include: determining the route of administration of the enteral nutrition; patient daily activity (e.g., steps per day); patient's ability to safely consume nutrition or hydration orally and if so, how much; goals of enteral nutrition (e.g., maintain, decrease, or increase body weight), times of day of normal eating and drinking; organic preference; and GI specific questions.

Based on the information inputted into the networked system, a nutrition plan is generated. In some embodiments, the provider documents a valid diagnosis for initiation of the enteral nutrition. The nutrition plan may include nutrition formulations to consider, recommendations for daily enteral nutrition and hydration volume, recommended feeding schedule (e.g., time and volume at breakfast, lunch, dinner, overnight). The nutrition plan may display the total recommended calories and breakdown of carbohydrates, proteins, fats, and micronutrients. The nutrition plan is then implemented with the wearable nutrition device 10. Routine monitoring for problems and progress toward nutritional and medical treatment goals are documented regularly. Potential problems with enteral nutrition are identified and preventative actions are taken. Principles of food safety are applied to preparation, storage, delivery, and administration of enteral formulas.

In some embodiments, the networked system 42 and wearable nutrition device 10 provides historical physiological and nutritional information. In some embodiments, that information is displayed to the user. In some embodiment, the display 34 is coupled to the module 30. In some embodiments, the display is wirelessly connected to the system. The information collected and monitored can include: weight, BMI, nutritional intake, calories burned, activity levels, and potential sources for future problems.

In some embodiments, the physiological and nutritional information can be displayed graphically. The stored physiological and nutritional information can also provide a repository for other measured or entered physiological parameters entered external to the wearable nutrition device 10. In some embodiments, the stored physiological and nutritional information is integrated with or includes information from other smart phone applications or repositories such as Apple Health Kit or other mobile health applications.

In some embodiments, the wearable nutrition device 10 directly senses the nutritional content of a packet (e.g., container 26) through a database and an identification code. In some embodiments, the wearable nutrition device 10 includes an accelerometer.

In some embodiments, a wired or wireless display indicates failure diagnostics. The system can identify metabolic problems (e.g., fluid, glucose, and electrolytes), gastrointestinal problems (e.g., nausea, vomiting, diarrhea, bloating, cramps, constipation) and mechanical problems (e.g., aspirational, tube clogging, tube migration, skin irritation).

In some embodiments, the wired or wireless display shows battery levels. In some embodiments, the wearable nutrition device 10 alerts the user through visual or auditory feedback.

In some embodiments, the wearable nutrition device 10 transmits and displays information into a web-browser or web-based portal view. In some embodiments, information from the wearable nutrition device 10 is displayed alongside other applicable measurements or conditions for diagnostic or therapeutic treatments.

In some embodiments, the networked system 42 stores physician and user entered data. In some embodiments, the networked system 42 stores nutritional flow rates. In some embodiments, the networked system stores information from one or more patients to assess patterns of use over time. In some embodiments, the networked system uses information stored overtime to assess physiological outcomes in certain scenarios.

### 8. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

With reference to Table 1, various types of data and information is contemplated for use with and by the system disclosed herein.

TABLE 1
Data for networked system

| Type | Data | Purpose |
|---|---|---|
| Device | Volume of | This value will help to track long-term feed- |
| Use | Fluid | ing trends that can inform better treatment |
| Data | Delivered per | decisions, identify problems with the |
| | Feed, Day | current regimen, and assist healthcare |
| | | providers with optimizing and personalizing |
| | | each patient's care. |
| | Number of | This value will help to track long-term |
| | Calories | feeding trends that can inform better |
| | Delivered | treatment decisions, identify problems |
| | per Feed, Day | with the current regimen, and assist |
| | | healthcare providers with optimizing and |
| | | personalizing each patient's care. |
| | Number of | This value will help to track long-term feed- |
| | Feeds | ting trends that can inform better treatment |
| | per Day, Week, | decisions, identify problems with the current |
| | Month, Year | regimen, and assist healthcare providers with |
| | | optimizing and personalizing each patient's |
| | | care. |
| | Rate of Fluid | These sensors will be able to track the |
| | Delivered per | number of rotations per unit time to |
| | Feed | allow for the calculation of the flow rate |
| | | being delivered to the patient. This will be |
| | | used to verify the input from the user and |
| | | ensure the device is operating within |
| | | the proper parameters. Through tracking |
| | | the rate of delivery, other important data can |
| | | be calculated using the type of formula |
| | | consumed by the user (i.e. calories |
| | | delivered, macronutrients consumed) |
| | Rate of Fluid | This value will help to track long-term |
| | Delivered, | feeding trends that can inform better |
| | Average | treatment decisions, identify problems |
| | (Daily, | with the current regimen, and assist |
| | Weekly, | healthcare providers with optimizing |
| | Monthly, | and personalizing each patient's |
| | Yearly) | care. This will also inform the next |
| | | generation of products to better serve the |
| | | observed use cases for optimization of |
| | | battery life, flow rate capabilities, and more. |
| | Type of | Through a passive detection such as NFC, or |
| | Nutritional | other, the placement of a reservoir or pouch |
| | Formula Used | would inform the system of the type of |
| | | formula, the calories per unit volume, and the |
| | | nutritional data associated with the formula. |
| | | This information is then used to approximate |
| | | volume, calories, and feeds delivered. |
| | Comprehensive | This will include tracking every feed that has |
| | Use Data | occurred, with their associated time, rate, |
| | | duration, calories and volume. This could |
| | | include each of the different formulas tried |
| | | by a given user, the length |
| | | of time the user has been feeding via |
| | | the pump, the total time the individual |
| | | has fed wearing vs not wearing the |
| | | system, and other long-term data values. |
| Individual | Disease State/ | This will comprise another data point in a |
| Patient | Indicated | machine learning algorithm to identify novel |
| Data | Condition for | trends among different types of patients and |
| | EN | their feed data. |
| | Health Care | This information will enable quick |
| | Provider | communications between the user's device |
| | | and their primary health care provider. This |
| | | will allow 1- or 2-way communication of |
| | | feeding data for better treatment decisions. |
| | Heart Rate/ | This sensor will provide the capabilities to |
| | Pulse | track blood oxygen levels (SpO2) as well as |
| | Oximetry | track overall heart rate through emission of a |
| | | specific wavelength of light (smart watches, |
| | | chest heart rate monitors). This is additional |
| | | data that can be paired with feed monitoring |
| | | data to provide further comprehensive |
| | | information to identify any trends around |
| | | feeding that may not be readily apparent. |
| | | This data will be fed into a machine |
| | | learning algorithm that will be able to |
| | | identify said trends and more. |
| | Approved EHR | This will provide the deepest and most |
| | Data | valuable data sets to enable a machine |
| | | learning algorithm to use a patient's entire |
| | | health history to identify possible trends |
| | | for improving their quality of life (QoL) |
| | | and their standard of care. |
| | Patient | This will be done by using the prescription, |
| | Compliance | provided by either the patient or healthcare |
| | Status | provider, and comparing it with the real-time |
| | | device statistics to determine how closely the |
| | | user is following the prescribed regimen. |
| | Demographic | This could include Age, Primary Diagnosis |
| | Information | (above), Locality, Mobility Status, Caregiver |
| | | Status (parent, professional, none). |
| | Activity Levels | This will serve to detect motion, to help the |
| | | device determine if a user is active (burning |
| | | calories), how many steps are taken, and |
| | | whether the user orientation is vertical or |
| | | horizontal during a feed. |
| Safety/ | Hang Time/ | This will be used to prevent any food safety |
| Risk | Time Since | hazards and ensure users are only consuming |
| Mitigation | Nutritional | formula that is within the window of safety. |
| Data | Formula | |
| | Opened | |
| | Internal Device | This will be used to ensure the motor does |
| | Temperature | not overheat and burnout, this will |
| | | additionally prevent the device from |
| | | becoming too hot on the user skin. |
| | Battery | This will provide user with daily information |
| | Capacity | of how much battery is left and when they |
| | per Charge | are required to recharge the device. |
| | Lifetime | This will provide comprehensive device |
| | Battery | health data to inform the responsible parties |
| | Cycles | when a device may require servicing or |
| | | replacement. |
| | Lifetime | This will provide comprehensive device |
| | Number of | health data to inform the responsible parties |
| | Drive Shaft | when a device may require servicing or |
| | Rotations | replacement. |
| | Pressure | This will serve to provide active monitoring |
| | Monitoring | of any possible occlusion or tube kinking |
| | | events that could interrupt or complicate the |
| | | feeding process. |
| | Individual | This will be comprehensive tracking of any |
| | Adverse Event | user inputs such as “journal entries”. |
| | Data | These will allow users to cite any adverse |
| | | events such as regurgitation, excessive gas |
| | | or bloating, gastric reflux, constipation etc. |
| | | In addition, any identified events such |
| | | as cease-flow, clogged or kinked tubing, |
| | | or misconnections will be automatically |
| | | tabulated into an adverse event tracker. |
| | Comprehensive | This will track the overall number of adverse |
| | Adverse Event | events during a given time period. This will |
| | Data | include the number of times an alarm is |
| | | triggered during a feed, the total time feeding |
| | | occurred (with and without interruptions), and |
| | | additionally tracking timestamps with each of |
| | | the individual adverse events. |
| Additional | Video, Audio, | This will enable individuals to perform |
| Data | Tele- | telehealth appointments through a mobile |
| Notes | conferencing | application with their enteral nutrition clinical |
| | Facilitating | providers. |
| | Information | |

### Example 2

With reference to FIGS. 2-4, example methods are illustrated. With reference to FIG. 2, a method includes (STEP 11) registering a health care provider in the networked portal (e.g., a web-based portal); (STEP 12) creating a provider account; and (STEP 13) populating provider account with pertinent information.

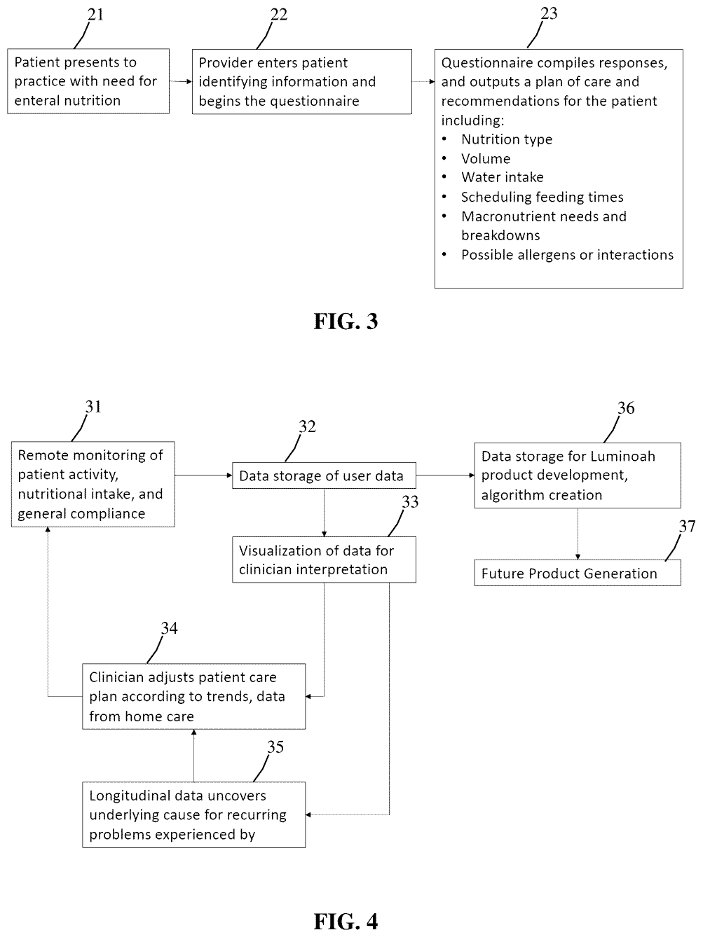

With reference to FIG. 3, a method includes (STEP 21) receiving a patient that presents to a provider's practice with an enteral nutrition need; (STEP 22) enter patient information into the networked portal; and (STEP 23) output a recommend nutrition plan.

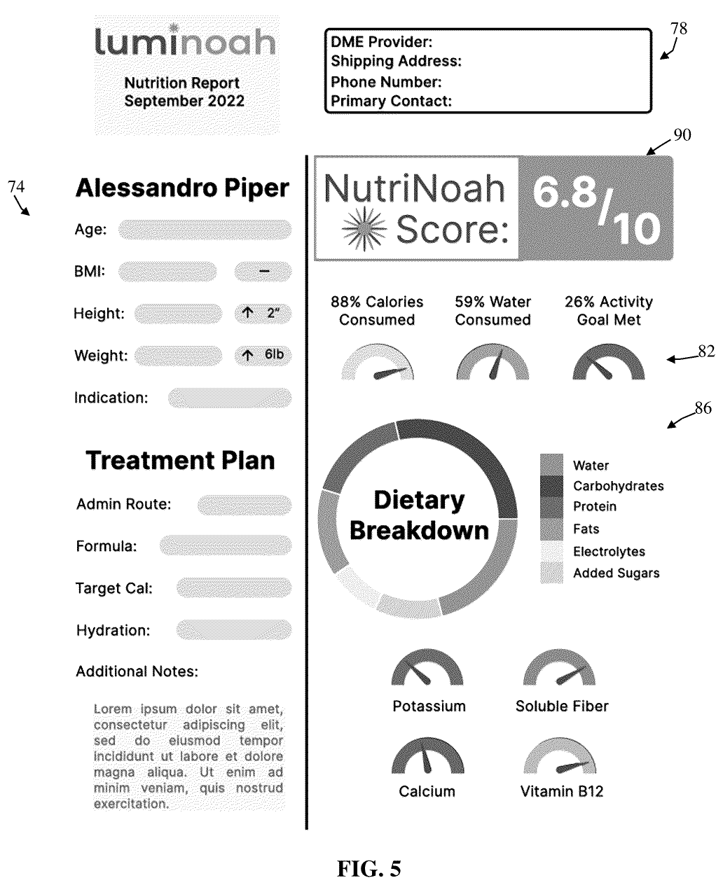

With reference to FIG. 4, a method includes (STEP 31) monitoring of patient activity, nutritional intake and nutrition plan compliance; and (STEP 32) storage of user data. The user data is utilized in (STEP 33) to visualize the data for interpretation. The method further includes (STEP 34) adjusting the nutrition plan based on real-time data or longitudinal data analysis (STEP 35). The method further includes (STEP 36) of storing and analyzing collected data for a plurality of users to develop predictive algorithms and improve product offerings (STEP 37).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art.

What is claimed is:

1. A method comprising:
inputting patient data;
generating a nutrition plan;
implementing the nutrition plan with a wearable nutrition device;
detecting a parameter with the wearable nutrition device; and
generating an updated nutrition plan based on the parameter.

2. The method of claim 1, further including implementing the updated nutrition plan with the wearable nutrition device.

3. The method of claim 1, wherein patient data includes gender, date of birth, height, weight, activity level, goal weight, organic preference, time preference, or any combination thereof.

4. The method of claim 1, wherein the parameter includes nutritional intake.

5. The method of claim 1, further including generating a nutrition report, wherein the nutrition report includes a historical view of patient nutrition.

6. The method of claim 5, wherein the nutrition report includes a score, and wherein the score is determined based on calories consumed, activity level, water consumed, micronutrition, patient demographic information, or any combination thereof.

7. The method of claim 5, wherein the nutrition report includes a score, and wherein the score is determined based on calories consumed, activity level, water consumed, micronutrition, and patient demographic information.

8. The method of claim 5, wherein the nutrition report is accessible via a network.

9. The method of claim 1, wherein generating the nutrition plan is based on a database of parameters from a plurality of users.

10. The method of claim 1, wherein the nutrition plan includes a custom formulation.

11. The method of claim 1, wherein the nutrition plan includes a caloric goal, a plurality of feed times, a flow rate, a formulation, or any combination thereof.

12. The method of claim 1, wherein the nutrition plan includes a caloric goal, a plurality of feed times, a flow rate, and a formulation.

13. The method of claim 1, wherein the patient data is collected by a wearable device.

14. The method of claim 1, further including detecting a second parameter with a wearable device; and generating the updated nutrition plan based on the second parameter.

15. The method of claim 1, wherein implementing the nutrition plan includes operating a motor to drive a pump of the wearable nutrition device, whereby a fluid is pumped from a container to a patient.

16. The method of claim 1, wherein detecting the parameter includes detecting an acceleration with an accelerometer positioned within the wearable nutrition device.

17. The method of claim 1, wherein detecting the parameter includes detecting an orientation with an accelerometer positioned within the wearable nutrition device.

18. The method of claim 1, wherein detecting the parameter includes receiving a user input from a user interface on the wearable nutrition device, and wherein the user input is in response to nausea, bloating, discomfort, regurgitation, dislodgment, an error message, or any combination thereof.

19. The method of claim 1, wherein detecting the parameter includes detecting with a gastric access device a pH, a microbiome, a gas, or any combination thereof.

20. The method of claim 1, further including displaying the parameter on a display of the wearable nutrition device.

21.-39. (canceled)
